(19) United States
(12) Patent Application Publication
(10) Pub. No.: US 2004/0220260 A1
(43) Pub. Date: Nov. 4, 2004

(54) Use of mono-or di-esters of cinnamic acid or of one of its derivatives and vitamin C, as donor of NO

(75) Inventor: Marie-Madeleine Cals-Grierson, Meudon (FR)

Correspondence Address: BURNS DOANE SWECKER & MATHIS L L P, POST OFFICE BOX 1404, ALEXANDRIA, VA, 22313-1404, US

(73) Assignee: L'OREAL S.A., Paris (FR)

(21) Appl. No.: 10/769,796

(22) Filed: Feb. 3, 2004

(30) Foreign Application Priority Data
Feb. 3, 2003 (FR) 03/01210

(51) Int. Cl. A61K 31/365

(52) U.S. Cl. 514/473

(57) ABSTRACT

The present invention relates to the use of mono- or di-esters of cinnamic acid or of one of its derivatives and vitamin C, in particular ascorbyl 2-O-cinnamate, to increase the production of nitric oxide in or on the skin or scalp or the mucous membranes as well as a procedure of application of nitric oxide in or on the skin, the scalp or the mucous membranes. It also relates to a cosmetic treatment procedure which makes use of these compounds as donors of NO.

### CROSS-REFERENCE TO PRIORITY/PROVISIONAL APPLICATIONS

This application claims priority under 35 U.S.C. § 119 of FR 03/01210, filed Feb. 3, 2003, and of provisional application Ser. No. 60/452,054, filed Mar. 6, 2003, both hereby expressly incorporated by reference and both assigned to the assignee hereof. This application is also a continuation of said '054 provisional.

The present invention relates to the use of mono- or di-esters of cinnamic acid or of one of its derivatives and vitamin C to increase the production of nitric oxide in or on the skin or the scalp or the mucous membranes as well as a procedure for the application of nitric oxide in or on the skin, the scalp or the mucous membranes.

In the field of health and cosmetics, the importance of nitric oxide NO is known in particular for the protective role of the NO produced by the nitric oxide synthases NOS (“Nitric Oxide Synthases”), of which there are three isoforms, NOS1 and NOS3 being constitutive and NOS2 inducible. NO is in fact implicated in many biological processes, in particular in the immune system, and it exhibits many interactions with the nucleic acids, the proteins, the low molecular weight thiols, etc.

Topical applications of nitric oxide have been described as well as complex compositions capable of generating it. Thus, the U.S. Pat. No. 6,103,275 describes compositions comprising two gels one of which contains a salt, like sodium nitrite, and the other contains ascorbic acid and maleic acid, the two gels being placed in contact before their application. The nitric oxide is generated by the resulting composition, directly on the skin.

However, these compositions possess disadvantages linked, in particular, to the mode of application and to the complex formulation for their implementation.

Consequently, it is noted that there exists a need for an active substance that can be used by the oral or topical route, and is efficacious for the production of NO in or on the skin, the scalp or the mucous membranes, in preventive or curative use, easy to formulate, apply or ingest, and lacking side effects.

The compounds of formula I below are described as antioxidants and in particular protectors with respect to the oxidation of the lipid constituents of the skin and described in pharmaceutical, cosmetic or foodstuff compositions in the document EP 664 290.

Surprisingly, the applicant has now demonstrated that these derivatives of mono- or di-esters of cinnamic acid or of one of its derivatives and vitamin C are particularly efficacious in producing nitric oxide in the skin, the mucous membranes or the scalp.

In fact, the applicant has been able to demonstrate a stimulant effect of these compounds on the production of NO, in the modulation screening test of the induction of NOS2 by normal human keratinocytes.

The products of the prior art known as donors of NO act by releasing the NO in situ, after a chemical reaction or enzymatic cleavage. The activity of these compounds would implicate a different mechanism, by increasing an endogenous route of production.

The subject of the invention is the use of mono- or di-ester derivatives of cinnamic acid or of one of its derivatives and vitamin C in order to enhance the production of nitric oxide in or on the skin, the mucous membranes or the scalp.

The compounds used in conformity with the invention to increase the production of nitric oxide in the skin or to obtain an efficacious production of it are the compounds of formula I.

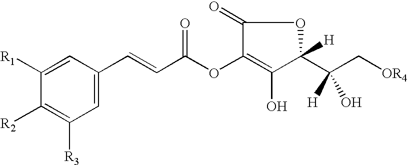

in which:

R₁, R₂ and R₃, independently of each other, represent H or an OH, alkoxy, fluoroalkoxy or alkylcarbonyloxy radical, and

R₄ represents H or —COR₅, R₅ representing a linear or branched C₁ to C₂₀ alkyl radical or the radical of the formula:

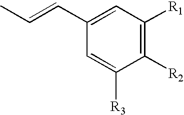

as well as related substances or substances capable of generating them, their analogues and precursors.

The alkoxy radical is preferably a methoxy, ethoxy or butoxy radical; the fluoroalkoxy radical is preferably the trifluoromethoxy radical and the alkylcarbonyl radical is preferably an acetoxy, propionyloxy or butyryloxy radical.

In a preferred manner, ascorbyl 2-O-cinnamate, ascorbyl 2-O-ferulate, ascorbyl 2-O-cafeate, ascorbyl 2-O-sinapate, 4′-acetoxy-ferulate of 2-O-[6-palmitoyl-ascorbyl] and in an even more preferred manner ascorbyl 2-O-cinnamate.

According to the invention, these compounds are used as active, cosmetic and/or pharmaceutical compounds to enhance the production of nitric oxide in or on the skin, the mucous membranes or the scalp, the application being carried out by the topical or oral route, in particular and the invention relates to the use of the above compounds for the preparation of compositions containing at least one of the compounds, acceptable salts or metal complexes and an acceptable excipient, designed for the treatment of the keratinic matter and mucous membranes, by the production of NO or by an increase of the production of NO.

Thus, these compounds can be used more particularly for the preparation of the compositions designed to promote the vascularisation of the skin or mucous membranes and/or the revascularisation of the areas representing psoriatic lesions and/or chronic ulcers, and/or to limit pruritus and/or eczema. In addition they can be used to regulate the growth of the skin bacteria, in particular to counteract the development of odour associated with sweat.

In addition, they can be used in compositions useful for protecting against the damage caused by ultraviolet light, like erythemas, and/or to promote melanogenesis and skin and/or hair pigmentation. Furthermore, they can be used to modulate the growth of hairs or the hair; for example in compositions to prevent hair loss.

They can also be used in cosmetic preparations for anti-ageing care designed to limit the deposit of highly cross-linked collagen and/or designed to promote the synthesis of collagen. Furthermore, they can be used in compositions designed to promote lipolysis and thus facilitate slimming. Moreover, they can be used in cosmetic compositions designed to modulate the pigmentation of the hair. They can also be used in cosmetic compositions useful for the regulation of sweating and the associated odours. They can also be used in cosmetic compositions useful for the regulation of fatty skins. Finally, they can be used in cosmetic compositions as myorelaxants.

In pharmaceutical applications the compounds of the invention are useful for preparing the compositions designed to combat skin mycoses and/or lupus erythematosus. The compounds can also be used for the preparation of pharmaceutical compositions useful for stimulating the microcirculation and for combating in particular the Raynaud syndrome and/or preventing or reducing epidermal proliferation in particular during scar formation. Finally, they can be used for the preparation of pharmaceutical compositions useful for regulating the immune system.

The cosmetic and pharmaceutical compositions contain in particular at least one compound of formula I or one of its acceptable salts or metal complexes in a cosmetically or pharmaceutically acceptable medium.

According to the invention, the compound(s) is/are present in these compositions in proportions ranging from 0.001 to 10% by weight.

The cosmetic and pharmaceutical compositions are available in various forms usually used in these pharmaceutical and cosmetic fields or in body hygiene, and in particular in the form of an ointment, cream, pomade, tablet, drinkable suspension, injection or gel, for example in the case of the pharmaceutical compositions and in the form of a gel, spray, lotion, emulsion or vesicular dispersion, for example, for the cosmetic compositions, as well as in the form of food supplements, without these particular forms being limiting for the cosmetic or pharmaceutical applications, respectively.

When the compounds of formula (I) are used in the context of a pharmaceutical treatment, the forms of administration can adopt the oral, topical, enteral or parenteral route, the pharmaceutically acceptable support depending on the form of administration chosen. The dosage is usually included between 1 and 100 mg/kg/day.

The cosmetically or pharmaceutically acceptable medium is a medium commonly used in everyday practice in the cosmetic or pharmaceutical field.

When the compounds of formula I are used in the context of a cosmetic treatment, the forms of application adopted are not only those of the topical route but also those of the oral route.

The invention will be better understood on reading the detailed description and the following examples.

The compounds of formula I can be prepared according to the procedures described in the document EP 664 290.

According to the invention, the topical route is preferred, involving direct application to the keratinic matter, like the skin, the scalp, the nails or the mucous membranes.

The compositions according to the invention are, however, available in all galenical and cosmetic forms compatible with their role as producer of NO, in particular the forms described in EP 664 290 or any other form, including that of food supplements.

These compositions are prepared according to the usual methods.

A cosmetically or dermatologically acceptable medium usually corresponds to a medium compatible with the keratinic matter, the skin, the scalp, the nails or the mucous membranes. The composition containing the active donor(s) of NO can thus be applied to the face, the neck, the hair and the nails, or any other skin region of the body (axillary, submammary, elbow folds, etc.).

By the topical route, the compositions according to the invention are available in particular in the form of aqueous, aqueous alcoholic or oily solutions, dispersions of the lotion or serum type, anhydrous or lipophilic gels, emulsions of liquid or semi-liquid consistency of the milk type, obtained by dispersion of a fatty phase in an aqueous phase (O/W) or the converse (W/O), or suspensions or emulsions of soft, semi-solid or solid consistency of the cream or gel type, or also microemulsions, microcapsules, microparticles or vesicular dispersions of the ionic and/or non-ionic type. These compositions are prepared according to the usual methods.

By the enteral route, the compositions according to the invention are available in the form of tablets, gelatine capsules, sugar-coated tablets, syrups, suspensions, solutions, microspheres or nanospheres or lipid or polymeric vesicles permitting controlled release.

By the parenteral route, the compositions are available in the form of solutions or suspensions for perfusion or for injection.

They can also be used for the scalp in the form of aqueous, alcoholic or aqueous alcoholic solutions, or in the form of creams, gels, emulsions, foams or also in the form of compositions for aerosols also containing a propellant under pressure.

The quantities of the different constituents of the compositions according to the invention are those used conventionally in the fields under consideration.

These compositions consist, in particular, of shaving foams, creams for the cleansing, protection, treatment or care of the face, the hands, the feet, the large anatomical folds or the body (for example, day creams, night creams, make-up removal creams, foundation make-up creams, anti-sun creams) fluid foundation make-up, make-up removal milks, body protection or care milks, anti-sun or better after-sun milks, lotions, gels or foams for skin care, like cleansing or disinfection lotions, anti-sun lotions, artificial tanning lotions, compositions for the bath, deodorising compositions containing a bactericidal agent, after-shave gels or lotions, depilatory creams, compositions against insect bites, anti-pain compositions or compositions for treating certain diseases in particular of the skin like those cited previously.

The compositions according to the invention may also consist of solid preparations consisting of disinfectant soaps or cakes of soap.

The compositions may also be packaged in the form of a composition for aerosols also containing a propellant under pressure.

The compounds used according to the invention can also be incorporated in various compositions for hair care or treatment, and in particular possibly antiparasitic shampoos, hair setting lotions, treating lotions, hairdressing creams or gels, hair dye compositions (in particular oxidation hair dyes), possibly in the form of rinses, restructuring lotions for the hair, compositions for the initial stage of a permanent waving, lotions or gels to prevent hair loss, etc.

The compositions of the invention may also be for buccal or dental use, for example as a toothpaste or a gargle. In this case, the compositions may contain the usual adjuvants and additives for the compositions for buccal use and in particular surfactants, thickeners, moisturisers, polishing agents such as silica, various active ingredients like the fluorides, in particular sodium fluoride, and possibly sweeteners like sodium saccharinate.

When the composition of the invention is an emulsion, the proportion of the fat phase may range from 5% to 80% by weight, and preferably from 5% to 50% by weight with respect to the total weight of the composition. The oils, the emulsifying agents and the co-emulsifying agents used in the composition in the form of an emulsion are chosen from those conventionally used in the cosmetic and pharmaceutical fields. The emulsifying agent and the co-emulsifying agent are present in the composition in a proportion ranging from 0.3% to 30% by weight, and preferably from 0.5 to 30% or better from 0.5 to 20% by weight with respect to the total weight of the composition. The emulsion may, in addition, contain lipid vesicles.

When the composition of the invention is a solution or an oily gel, the fat phase may represent more than 90% of the total weight of the composition.

In a known manner, the composition of the invention may also contain adjuvants usually used in the cosmetic or pharmaceutical field, such as hydrophilic or lipophilic gelling agents, hydrophilic or lipophilic active compounds, preservatives, antioxidants, solvents, perfumes, fillers, filters, bactericidal agents, odour absorbers and colouring matters. The quantities of these various adjuvants are those conventionally used in the cosmetic or pharmaceutical field, and for example vary from 0.01% to 10% of the total weight of the composition. These adjuvants, depending on their nature, maybe introduced in the fat phase, in the aqueous phase and/or in lipid spherules.

As oils usable in the invention, mention may be made of mineral oils (vaseline oils), vegetable oils (liquid fraction of karite butter, sunflower oil), animal oils (perhydrosqualene), synthetic oils (Purcellin oil), siliconised oils (cyclomethicone) and fluorinated oils (perfluoropolyethers). Fatty alcohols, fatty acids (stearic acid), waxes (paraffin, carnauba, beeswax) can also be used as fatty materials.

As emulsifying agents usable in the invention, mention may be made for example of glycerol stearate, polysorbate 60 and the PEG-6/PEG-32/glycol stearate mixture sold under the trade name Tefose® 63 by the Gattefosse company.

As solvents usable in the invention, mention may be made of the lower alcohols, in particular ethanol, isopropanol and propylene glycol.

As hydrophilic gelling agents, mention may be made of carboxyvinyl polymers (carbomer), the acrylic copolymers such as the acrylate/alkylacrylate copolymers, the polyacrylamides, the polysaccharides such as hydroxypropylcellulose, natural gums and clays and, as lipophilic gelling agents, mention may be made of the modified clays like the bentones, the metal salts of fatty acids like the aluminium stearates and hydrophobic silica or also ethylcellulose, polyethylene.

The compounds used according to the invention may be used in combination with other active compounds.

As hydrophilic active compounds, the proteins or protein hydrolysates, the amino acids, the polyols, urea, allantoin, the sugars and sugar derivatives, the water-soluble vitamins, starch and plant extracts, in particular those of aloe vera, for example, may be used.

As lipophilic active compounds, retinol (vitamin A) and its derivatives, tocopherol (vitamin E) and its derivatives, the essential fatty acids, the ceramides, the essential oils or the polyphenols, for example, may be used.

In addition, it is possible to combine the NO donor compounds according to the invention with other active NO donor agents or active agents designed in particular for the prevention and/or treatment of cutaneous, mucosal and/or hair infections.

Among these active agents, mention may be made as examples of the N-arylmethyleneethylenediaminetriacetates, N-arylmethylene-iminodiacetates or N,N′-diarylmethylene ethylenediamineacetates and their salts and metal complexes and in a preferred manner of N,N′-bis-(3,4,5-trimethoxybenzyl)ethylenediamine-diacetic acid, its analogues and precursors. As salts, mention maybe made of the addition salts with a mineral acid like the acids H₂SO₄, HCl, HNO₃ or H₃PO₄, for example, and the addition salts with a mineral base like NaOH or KOH. As metal complexes, mention may be made of the complexes formed by the addition of ZnCl₂ or CaCl₂, for example.

Moreover, combinations with other NO donors can be made. Among the latter, for example, the nitro and S-nitroso derivatives of non-steroidal anti-inflammatory agents (NO-NSAIDs) and steroids (NO-steroids), usually obtained by the addition of an ONO₂ group through an ester linkage to the parent anti-inflammatory molecule. These compounds are known under the designations NO-aspirin, NO-paracetamol, NO-fulbiprofen, for example.

More generally, direct NO donors or NO donors requiring metabolism for the production of NO, irrespective of whether their mechanism of action implicates endogenous or exogenous donor activity, may also be combined with the compounds used according to the invention.

In addition, the subject of the present invention is a cosmetic treatment procedure which makes use of the above compound(s) or the cosmetic composition, in particular for the uses described above. It is possible to apply to the skin, the mucous membranes and/or the scalp the cosmetic compositions or even ingest compositions designed for that purpose.

Preferentially, the cosmetic treatment procedure consists of applying to the skin, the scalp and/or the mucous membranes, a composition such as that described above. Thus, the cosmetic treatment procedure of the invention can be implemented in particular by applying the hygienic or cosmetic compositions such as defined above, according to the usually procedure for the use of these compositions and for example: application of creams, gels, serums, lotions, make-up removal milks or after-sun compositions to the skin or wet hair, shampoos or even application of toothpaste to the gums.

### EXAMPLE 1

### Biological Activity of Ascorbyl 2-O-cinnamate

The activity of ascorbyl 2-O-cinnamate on the inducible NO synthase was evaluated in the assay described by Heck et al. (J.B.C., vol. 267, No. 30, 21277-21280, 25 Oct. 1992), a screening test of the modulation of induction of NOS2 by normal human keratinocytes. The objective of this test is, briefly, to evaluate the concentration of nitrate and nitrite after stimulation of the NO-synthase 2.

The test is carried out on a culture of normal human keratinocytes, derived from samples taken. The induction of the inducible NO synthase (NOS2) was caused by the addition of a combination of several cytokines to the culture medium. The product tested was applied at three concentrations varying from 10 to 1000 μM.

The product tested was classed in one of the following two categories: inhibitor (I) or donor (D) of NO (nitric oxide) according to its capacity to diminish or increase, respectively, the quantity of nitrites and nitrates produced compared to the control signal (without cytokines).

The following controls were introduced in the test:

A: positive control (induction of the enzyme): mixtures of interferon-γ (1000 u/ml) and interleukin 1-β (100 u/ml)

B: negative control (maximal inhibition): N⁹-monomethyl-L-arginine. (L form) at 200 μm;

C: control of specificity of the inhibition: N⁹-monomethyl-L-arginine (D form) at 200 μm;

In order to determine the activity of the test product the quantity of stable NO reaction products (nitrites and nitrates) is measured with the aid of the “nitric calorimetric assay” kit sold by the Boehringer company under catalogue number 1756.28.

Ascorbyl 2-O-cinnamate was tested at concentrations of 100 μM, 500 μM and 1000 μM in ethanol

| Produit tested | % inhibition |
|---|---|
| A | 0 |
| B | 100 |
| C | 0 |
| Ascorbyl 2-O-cinnamate: 100 μM | 24.79% |
| Ascorbyl 2-O-cinnamate: 500 μM | 53.74% |
| Ascorbyl 2-O-cinnamate: 1000 μM | 74.4% |

It is concluded that ascorbyl 2-O-cinnamate exhibits a NO donor effect.

Moreover, the cytotoxicity was expressed as % diminution of the signal compared with the control. A product was considered to be not very cytotoxic from 0-20%, acceptable up to 40% and not retained for values >40%.

It was also concluded that the product is not toxic.

3. Use of at least one compound of formula I 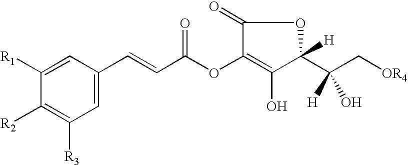
in which:
R₁, R₂ and R₃ independently represent H or a OH, alkoxy, fluoroalkoxy or alkylcarbonyloxy radical, and
R₄ represents H or —COR₅, R₅ representing a linear or branched C₁ to C₂₀ alkyl radical or the radical of formula: 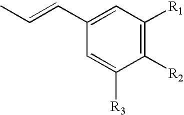
for the preparation of a composition comprising at least one compound of formula I and an acceptable excipient, designed for the treatment of keratinic matter and mucous membranes by the production of nitric oxide or by an increase of the production of nitric oxide in or on the skin, the mucous membranes or the scalp.

2. Use according to claim 1, characterized in that the composition is useful for promoting the vascularisation of the skin or mucous membranes.

3. Use according to one of the claims 1 to 2, characterized in that the composition is useful for revascularising psoriatic lesions and/or chronic ulcers.

4. Use according to one of the claims 1 to 3, characterized in that the composition is useful for limiting pruritus and/or eczema.

5. Use according to one of the claims 1 to 4, characterized in that the composition is useful for protecting the damage caused by ultraviolet radiation, like erythemas.

6. Use according to one of the claims 1 to 5, characterized in that the composition is useful for modulating the growth of hairs or the hair.

7. Use according to one of the claims 1 to 6, characterized in that the composition is useful for promoting melanogenesis and skin and/or hair pigmentation.

8. Use according to one of the claims 1 to 7, characterized in that the composition is used to regulate the growth of skin bacteria.

9. Use according to one of the claims 1 to 8, characterized in that the composition is cosmetic and designed to limit hair loss.

10. Use according to one of the claims 1 to 9, characterized in that the cosmetic composition is useful for modulating the pigmentation of the hair.

11. Use according to one of the claims 1 to 10, characterized in that the cosmetic composition is an anti-age care composition designed to promote the synthesis of collagen and/or designed to limit the deposit of highly cross-linked collagen.

12. Use according to one of the claims 1 to 11, characterized in that the cosmetic composition is useful in preparations for promoting lipolysis.

13. Use according to one of the claims 1 to 12, characterized in that the cosmetic composition is useful for promoting the regulation of fatty skins.

14. Use according to one of the claims 1 to 13, characterized in that the cosmetic composition is useful for promoting the regulation of sweating and associated odours.

15. Use according to one of the claims 1 to 14, characterized in that the composition is used in cosmetic applications as a myorelaxant.

16. Use according to one of the claims 1 to 8, characterized in that the pharmaceutical composition is useful in a treatment to combat skin mycoses.

17. Use according to one of the claims 1 to 8 and 16, characterized in that the pharmaceutical composition is useful in a treatment to combat lupus erythematosis.

18. Use according to one of the claims 1 to 8 and 16 or 17, characterized in that the pharmaceutical composition is useful in a treatment to stimulate the microcirculation and/or combat the Raynaud syndrome.

19. Use according to one of the claims 1 to 8 and 16 to 18, characterized in that the pharmaceutical composition is useful in a treatment to prevent or reduce epidermal proliferation.

20. Use according to one of the claims 1 to 8 and 16 to 19, characterized in that the pharmaceutical composition is useful in a treatment to regulate the immune system.

21. Use according to one of the claims 1 to 8 and 16 to 20, characterized in that the pharmaceutical composition is useful for limiting hair loss and for promoting its regrowth.

22. Use according to one of the claims 1 to 21, characterized in that the product of formula I is ascorbyl 2-O-cinamate.

23. Use for the cosmetic treatment of the organism, for the purpose of producing or increasing the production of nitric oxide in or on the skin, the hair or the mucous membranes of a compound of formula I defined in claim 1 or 22.

24. Use as NO donor of a compound of formula I defined in claim 1 or 22.

25. Cosmetic treatment procedure comprising applying to the skin, the mucous membranes and/or the scalp or of ingesting a composition containing at least one compound of formula I defined in claim 1.

26. Procedure according to claim 25, in which the composition contains at least ascorbyl 2-O-cinnamate.
